(19) United States
(12) Patent Application Publication
(10) Pub. No.: US 2020/0118647 A1
(43) Pub. Date: Apr. 16, 2020

(54) PHENOTYPE TRAIT PREDICTION WITH THRESHOLD POLYGENIC RISK SCORE

(71) Applicant: Ancestry.com DNA, LLC, Lehi, UT (US)

(72) Inventors: Miao Zhang, Burlingame, CA (US); Julie M. Granka, San Francisco, CA (US); Thi Hong Luong Nguyen, San Bruno, CA (US); Ahna R. Girshick, Berkeley, CA (US); Hamutal Arbel, San Francisco, CA (US)

(21) Appl. No.: 16/598,988

(22) Filed: Oct. 10, 2019

Related U.S. Application Data
(60) Provisional application No. 62/745,245, filed on Oct. 12, 2018.
(60) Provisional application No. 62/879,073, filed on Jul. 26, 2019.

(51) Int. Cl. G16B 40/00; G16B 20/20; G16B 30/00; G06N 20/10; C12Q 1/6827

(52) U.S. Cl. CPC G16B 40/00; G16B 20/20; C12Q 1/6827; G06N 20/10; G16B 30/00

(57) ABSTRACT

A system and method predicts a phenotypic trait for an individual. The system identifies a subset of SNP loci with predictive ability of the phenotypic trait. The system calculates a PRS for the individual based on the individual's genetic dataset at the identified subset of SNP loci. The system compares the PRS to a threshold PRS. The threshold PRS is determined by calculating, for each training individual of a plurality of training individuals including some reported to have and some reported to not have the phenotypic trait, a PRS, sweeping through a domain of PRS while calculating a true positive rate and a false positive rate, and then identifying an optimal threshold PRS as the threshold PRS. The system generates a prediction whether the individual has the phenotypic trait based on the comparison.

### CROSS-REFERENCE TO RELATED APPLICATIONS

The present application claims the benefit of U.S. Provisional Patent Application No. 62/745,245 filed on Oct. 12, 2018, and U.S. Provisional Patent Application No. 62/879,073 filed on Jul. 26, 2019, which are hereby incorporated by reference in their entirety.

### FIELD

The disclosed embodiments relate to assessing populations in which variants of interest may have arisen and propagated and discovering historical populations from the pattern of genetic relationships between people.

### BACKGROUND

Although humans are, genetically speaking, almost entirely identical, small differences in human DNA are responsible for some observed genetic variation between individuals. Genetic variations manifest visually in variance of phenotypic traits among the human population. The human genome mutation rate is estimated to be 1.1*10\hat{ }−8 per site per generation. This leads to a variant approximately every 300 base pairs. Most of the mutations that are passed down to descendants are related to single-nucleotide polymorphism (SNP). SNP is a substitution of a single nucleotide that occurs at a specific locus in the genome. Learning about population structure from genetic polymorphism data is an important topic in genetics.

### SUMMARY

Disclosed herein relates to a method and/or a system for predicting a phenotypic trait in an individual by considering SNP variation in the individual. The system receives a genetic dataset for the individual, the genetic dataset including a plurality of reads at a plurality of SNP loci. The reads may indicate the individual's genotype at each SNP locus. The system identifies a subset of the plurality of SNP loci with predictive ability of the phenotypic trait, e.g., by conducting a genome-wide association study (GWAS) for the phenotypic trait and identifying SNP loci having a p-value score below a certain threshold. The system calculates a polygenic risk score (PRS) for the individual based on the genetic dataset of the individual at the identified subset of SNP loci with a PRS function. The PRS function may also weight the SNP loci and/or normalize the PRS. The system compares the polygenic risk score to a threshold polygenic risk score and generates a prediction of whether the individual likely has the phenotypic trait based on the comparison. In some embodiments, the phenotypic trait may include a plurality of labels, wherein the system determines multiple PRS thresholds to generate predictions between the varying labels.

The system determines the threshold polygenic risk score with evaluating a receiver operating characteristic (ROC) curve of PRS for a group of training individuals. The system obtains genetic datasets for the training individuals. Among the training individuals, some reported to have the phenotypic trait and others reported to not have the phenotypic trait. In particular embodiments, the training individuals share one or more common characteristics, e.g., sex, age, ethnicity, genetic community, etc. For each training individual, the system calculates a PRS. The system sweeps through a domain of the PRS, i.e., a range of candidate threshold PRS, and calculates a true positive rate and a false positive rate. The system may plot the true positive rate to the false positive rate as a function of PRS. The system determines the threshold PRS that optimally maximizes the true positive rate while minimizing the false positive rate.

In alternate embodiments, the system implements a machine learning model to predict whether an individual has a phenotypic trait. The system uses a similar process to identify a subset of SNP loci with high predictive ability of identifying the phenotypic trait. The system generates feature vectors for training individuals that include the genotype of the training individual at each SNP locus in the identified subset. Knowing how each training individual reported, the system trains the machine learning model with the training individuals. The trained machine learning model inputs a feature vector comprising genetic data at the identified subset of SNP loci and outputs a prediction of a likelihood the individual likely has the phenotype. In these embodiments with the machine learning model, the machine learning model may be implemented as a binary classifier with phenotypic traits that have binary labels or as a multiclass classifier with phenotypic traits that have a plurality of labels. In other embodiments, the machine learning model may be implemented as a regression for a phenotypic trait that is continuous, e.g., height.

### BRIEF DESCRIPTION OF THE DRAWINGS

FIG. 1 illustrates a diagram of a system environment of an example computing system, in accordance with an embodiment.

FIG. 2 is a block diagram of an architecture of an example computing system, in accordance with an embodiment.

FIG. 3 is a block diagram of an architecture of an example phenotype prediction engine, in accordance with an embodiment.

FIG. 4 is a graph of SNP loci predictive of male pattern baldness in a cohort of individuals of European ancestry, in accordance with an embodiment.

FIG. 5 is a graph of predictive sensitivity over a domain of polygenic risk score for four different subsets of SNP loci, in accordance with an embodiment.

FIG. 6A is a flowchart illustrating a process of identifying a subset of SNP loci for consideration in predicting a phenotypic trait, in accordance with an embodiment.

FIG. 6B is a flowchart illustrating a process of identifying a threshold polygenic risk score for determining whether an individual has a phenotypic trait, in accordance with an embodiment.

FIG. 7 is a flowchart illustrating a process of predicting whether an individual has a phenotypic trait using PRS, in accordance with an embodiment.

FIG. 8 is a flowchart illustrating a process of predicting whether an individual has a phenotypic trait using a machine learning model, in accordance with an embodiment.

FIG. 9 is a block diagram of an example computing device, in accordance with an embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### Example System Environment

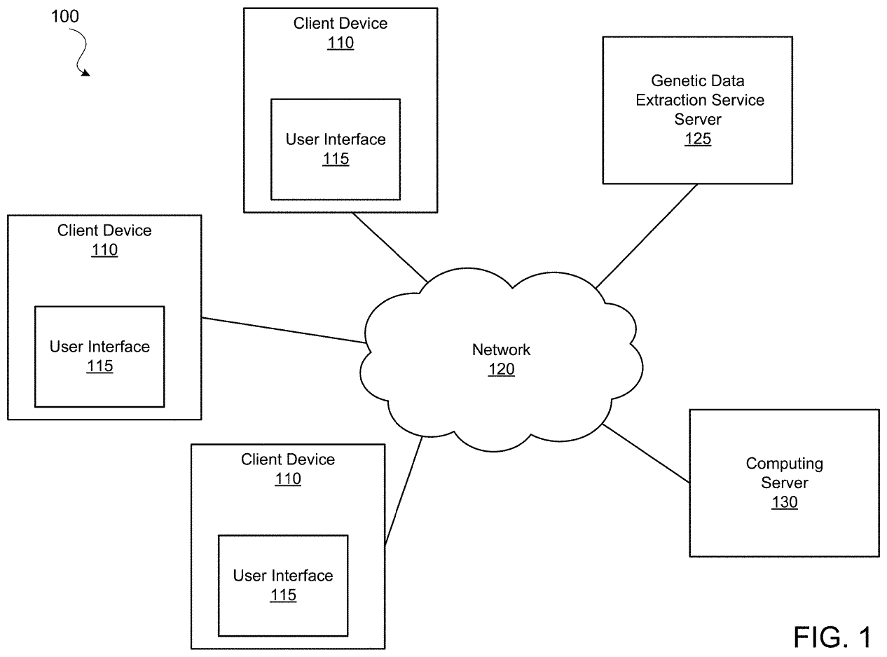

FIG. 1 illustrates a diagram of a system environment 100 of an example computing server 130, in accordance with an embodiment. The system environment 100 shown in FIG. 1 includes one or more client devices 110, a network 120, a genetic data extraction service server 125, and a computing server 130. In various embodiments, the system environment 100 may include fewer or additional components. The system environment 100 may also include different components.

The client devices 110 are one or more computing devices capable of receiving user input as well as transmitting and/or receiving data via a network 120. Example computing devices include desktop computers, laptop computers, personal digital assistants (PDAs), smartphones, tablets, wearable electronic devices (e.g., smartwatches), smart household appliance (e.g., smart televisions, smart speakers, smart home hubs), Internet of Things (IoT) devices or other suitable electronic devices. A client device 110 communicates to other components via the network 120. Users may be customers of the computing server 130 or any individuals who access the system of the computing server 130, such as an online website or a mobile application. In one embodiment, a client device 110 executes an application that launches a graphical user interface (GUI) for a user of the client device 110 to interact with the computing server 130. The GUI may be an example of a user interface 115. A client device 110 may also execute a web browser application to enable interactions between the client device 110 and the computing server 130 via the network 120. In another embodiment, the user interface 115 may take the form of a software application published by the computing server 130 and installed on the user device 110. In yet another embodiment, a client device 110 interacts with the computing server 130 through an application programming interface (API) running on a native operating system of the client device 110, such as IOS or ANDROID.

The network 120 provides connections to the components of the system environment 100 through one or more sub-networks, which may include any combination of local area and/or wide area networks, using both wired and/or wireless communication systems. In one embodiment, a network 120 uses standard communications technologies and/or protocols. For example, a network 120 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 120 may be represented using any suitable format, such as hypertext markup language (HTML) or extensible markup language (XML). In some embodiments, all or some of the communication links of a network 120 may be encrypted using any suitable technique or techniques such as secure sockets layer (SSL), transport layer security (TLS), virtual private networks (VPNs), Internet Protocol security (IPsec), etc. The network 120 also includes links and packet switching networks such as the Internet.

Individuals, who may be customers of a company operating the computing server 130, provide biological samples for analysis of their genetic data. In one embodiment, an individual uses a sample collection kit to provide a biological sample (e.g., saliva, blood, hair, tissue) from which genetic data is extracted and determined according to nucleotide processing techniques such as amplification and sequencing. Amplification may include using polymerase chain reaction (PCR) to amplify segments of nucleotide samples. Sequencing may include sequencing of deoxyribonucleic acid (DNA) sequencing, ribonucleic acid (RNA) sequencing, etc. Suitable sequencing techniques may include Sanger sequencing and massively parallel sequencing such as various next-generation sequencing (NGS) techniques including whole genome sequencing, pyrosequencing, sequencing by synthesis, sequencing by ligation, and ion semiconductor sequencing. Genetic data extraction service server 125 receives biological samples from users of the computing server 130. The genetic data extraction service server 125 performs sequencing of the biological samples and determines the base pair sequences of the individuals. The genetic data extraction service server 125 generates the genetic data of the individuals based on the sequencing results. The genetic data may include data sequenced from DNA or RNA and may include base pairs from coding and/or noncoding regions of DNA.

The genetic data may take different forms. For example, in one embodiment, the genetic data may be the base pair sequence of an individual. The base pair sequence may include the whole genome or a part of the genome such as certain genetic loci of interest. In another embodiment, the genetic data extraction service server 125 may determine genotypes from sequencing results, for example by identifying genotype values of single nucleotide polymorphisms (SNPs) present within the DNA. The results in this example may include a sequence of genotypes corresponding to various SNP loci. In one embodiment, the genetic data extraction service server 125 may perform data pre-processing of the genetic data to convert raw sequences of base pairs to sequences of genotypes at target SNP loci. Since a typical human genome may differ from a reference human genome at only several million SNP loci (as opposed to billions of base pairs in the whole genome), the genetic data extraction service server 125 may extract only the genotypes at a set of target SNP loci and transmit the extracted data to the computing server 130 as the genetic dataset of an individual.

The computing server 130 performs various analysis of the genetic data, genealogical data, and users' survey responses to generate results regarding the phenotypes and genealogy of users of computing server 130. Depending on the embodiments, the computing server 130 may also be referring to as an online server, a personal genetic service server, a genealogy server, a family tree building server, and/or a social networking system. The computing server 130 receives genetic data from the genetic data extraction service server 125 and stores the genetic data in the data store of the computing server 130. The computing server 130 may analyze the data to generate results regarding the genetics or genealogy of users. The results regarding the genetics or genealogy of users may include the ethnic compositions of users, paternal and maternal genetic analysis, potential family relatives, ancestor information, analyses of DNA data, potential or identified phenotypes of users (e.g., diseases, traits, and other characteristics), etc. The computing server 130 may present or cause the user interface 115 to present the results to the users through a GUI displayed at the client device 110. The results may include graphical elements, textual information, data, charts, and other elements such as family trees.

In one embodiment, the computing server 130 also allows various users to create one or more genealogical profiles of the user. The genealogical profile may include a list of individuals (e.g., ancestors, relatives, friends, and other people of interest) who are added or selected by the user or suggested by the computing server 130 based on the genealogical records and/or genetic records. The user interface 115 controlled by or in communication with the computing server 130 may display the individuals in a list or as a family tree such as in the form of a pedigree chart. In one embodiment, subject to user's privacy setting and authorization, the computing server 130 may allow the user's genetic dataset to be linked to the user profile and to one or more of the family trees. The users may also authorize the computing server 130 to analyze their genetic dataset and allow their profiles to be discovered by other users.

### Example Computing Server Architecture

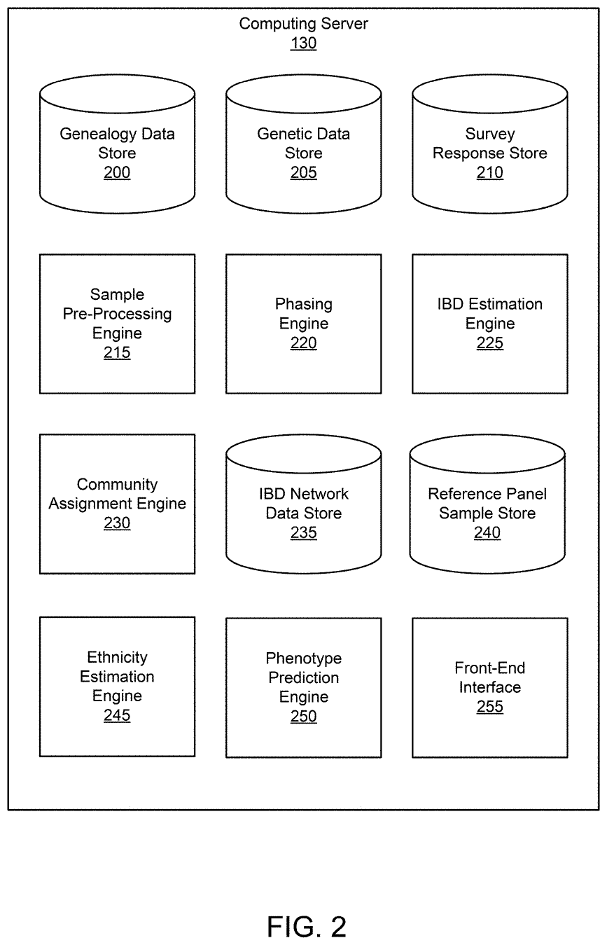

FIG. 2 is a block diagram of an architecture of an example computing server 130, in accordance with an embodiment. In the embodiment shown in FIG. 2, the computing server 130 includes a genealogy data store 200, a genetic data store 205, a survey response store 210, a sample pre-processing engine 215, a phasing engine 220, an IBD estimation engine 225, a community assignment engine 230, an IBD network data store 235, a reference panel sample store 240, an ethnicity estimation engine 245, and a front-end interface 255. The functions of the computing server 130 may be distributed among the elements in a different manner than described. In various embodiments, the computing server 130 may include different components and fewer or additional components. Each of the various data stores may be a single storage device, a server controlling multiple storage devices, or a distributed network that is accessible through multiple nodes (e.g., a cloud storage system).

The computing server 130 stores various data of different individuals, including genetic data, genealogical data, and survey response data. The computing server 130 processes the genetic data of users to identify shared identity-by-descent (IBD) segments between individuals. The genealogical data and survey response data may be part of user profile data. The amount and type of user profile data stored for each user may vary based on the information of a user, which is provided by the user as she creates an account and profile at a system operated by the computing server 130 and continues to build her profile, family tree, and social network at the system and to link her profile with her genetic data. Users may provide data via the user interface 115 of a client device 110. Initially and as a user continues to build her genealogical profile, the user may be prompted to answer questions related to basic information of the user (e.g., name, date of birth, birthplace, etc.) and later on more advanced questions that may be useful for obtaining additional genealogical and survey data. The computing server 130 may also include survey questions regarding various traits, characteristics, preferences, habits, lifestyle, environment, etc. of the users.

Genealogical data may be stored in the genealogical data store 200 and may include various types of data that are related to tracing family relatives of users. Examples of genealogical data include names (first, last, middle, suffixes), gender, birth locations, date of birth, date of death, marriage information, spouse's information kinships, family history, dates and places for life events (e.g., birth and death), other vital data, and the like. In some instances, family history can take the form of a pedigree of that individual (e.g., the recorded relationships in the family). The family tree information associated with a user includes one or more specified nodes. Each node in the family tree represents the individual, an ancestor of the individual who might have passed down genetic material to the individual, and the individual's other relatives including siblings, cousins, offspring in some cases. Genealogical data may also include connections and relationships among users of the computing server 130. The information related to the connections among a user and her relatives that may be associated with a family tree may also be referred to as pedigree data or family tree data.

In addition to user-input data, genealogical data may also take other forms that are obtained from various sources such as public records and third-party data collectors. For example, genealogical records from public sources include birth records, marriage records, death records, census records, court records, probate records, adoption records, obituary records, etc. Likewise, genealogical data may include data from one or more of a pedigree of an individual, the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, a motor vehicle database, and the like.

Furthermore, the genealogical data store 200 may also include relationship information inferred from the genetic samples stored in the genetic data store 205 and information received from the individuals. For example, the relationship information may include which individuals are genetically related, how they are related, how many generations back they share common ancestors, lengths and locations of IBD segments shared, which genetic communities an individual is a part of, variants carried by the individual, and the like.

The computing server 130 maintains genetic datasets of individuals in the genetic data store 205. A genetic dataset of an individual may be a digital dataset of nucleotide data and corresponding metadata. A genetic dataset may contain data of the whole or portions of an individual's genome. The genetic data store 205 may store a pointer to a location associated with the genealogical data store 200 associated with the individual. A genetic dataset may take different forms. In one embodiment, a genetic dataset may take the form of a base pair sequence of the sequencing result of an individual. A base pair sequence dataset may include the whole genome of the individual (e.g., obtained from a whole-genome sequencing) or some parts of the genome (e.g., genetic loci of interest).

In another embodiment, a genetic dataset may take the form of sequences of genetic markers. Examples of genetic markers may include target SNP loci (e.g., allele loci) filtered from the sequencing results. A SNP locus may be associated with a unique identifier. The genetic dataset may be in a form of a diploid data that include a sequencing of genotypes, such as genotypes at the target SNP loci, or the whole base pair sequence that includes genotypes at known SNP loci that vary between individuals and other base pair sites that are not commonly associated with known SNP loci. The diploid dataset may be referred to as a genotype dataset or a genotype sequence. Genotype may have a different meaning in various context. In one context, an individual's genotype may refer to a collection of diploid alleles of an individual. In other contexts, a genotype may be a pair of alleles present on two chromosomes for an individual at a given genetic marker such as a SNP locus.

A genotype at a SNP locus may include a pair of alleles. The pair of alleles may be homozygous (e.g., A-A or G-G) or heterozygous (e.g., A-T, C-T). Instead of storing the actual nucleotides, the genetic data store 205 may store genetic data that are converted to bits. For a given SNP locus, oftentimes only two nucleotide alleles (instead of all 4) are observed. As such, a 2-bit number may represent the genotype at a SNP locus. For example, “00” may represent homozygous first alleles, “11” may represent homozygous second alleles, and “01” or “10” may represent heterozygous alleles. A genotype at a SNP locus has the SNP if at least one of the alleles includes the SNP. For example, both genotypes of A-A and A-T have the SNP if the unmutated genotype without the SNP is T-T. A separate library may store what nucleotide corresponds to the first allele and what nucleotide corresponds to the second allele at a given SNP locus.

A diploid dataset may also be phased into two sets of haploid data, one corresponding to a first parent side and another corresponding to a second parent side. The phased datasets may be referred to as haplotype datasets or haplotype sequence.

The computing server 130 may present various survey questions to its users from time to time. The responses to the survey questions may be stored at survey response store 210. The survey questions may be related to various aspects of the users and the users' family. Some survey questions may be related to users' phenotypes, while other may be related to environmental factors of the users.

For example, survey questions may concern health or disease-related phenotypes, such as questions related to the presence or absence of genetic diseases or disorders, inheritable diseases or disorders, or other common diseases or disorders that have family history as one of the risk factors, questions regarding any diagnosis of increased risk of any diseases or disorders, and questions concerning wellness-related issues such as family history of obesity, family history of causes of death, etc. The diseases identified by the survey questions may be related to single-gene diseases or disorders that are caused by a single-nucleotide variant, an insertion, or a deletion. The diseases identified by the survey questions may also be multifactorial inheritance disorders that may be caused by a combination of environmental factors and genes. Example of multifactorial inheritance disorders may include heart disease, Alzheimer's diseases, diabetes, cancer, and obesity. The computing server 130 may obtain data of a user's disease-related phenotypes from survey questions of health history of the user and her family and also from health records uploaded by the user.

Survey questions also may be related to other types of phenotypes such as traits and characteristics of the users. A survey regarding traits and characteristics may include questions related to eye color, iris pattern, freckles, chin types, finger length, dimple chin, earlobe types, hair color, hair curl, skin pigmentation, susceptibility to skin burn, bitter taste, male baldness, baldness pattern, presence of unibrow, presence of wisdom teeth, height, and weight. A survey regarding traits and characteristics also may include questions related to users' taste and smell such as the ability to taste bitterness, asparagus smell, cilantro aversion, etc. A survey regarding traits and characteristics may further include questions related to users' lactose tolerance, caffeine consumption, malaria resistance, norovirus resistance, muscle performance, alcohol flush, etc.

Computing server 130 also may present various survey questions to its users related to users' preferences, habits and lifestyle. For example, a survey regarding users' preferences may include questions related to things and activities that user like or dislike, such as whether a user enjoy ice cream, egg, music, a certain sport, video games, etc. A survey related to habits and lifestyle may include question regarding smoking habits, alcohol consumption and frequency, daily exercise duration, sleeping cycles and problems, hobbies, and traveling preferences.

The survey response data, the genetic data, and the genealogical data may subject to the privacy and authorization setting from the users. For example, when presented with a survey question, a user may select to answer or skip the question. The computing server 130 may present users from time to time information regarding users' selection of the extent of information and data shared. The computing server 130 also may maintain and enforce one or more privacy settings for users in connection with the access of the user data. For example, the user may pre-authorize the access of the data and may change the setting as wish. The privacy settings also may allow a user to specify (e.g., by opting out, by not opting in) whether the computing server 130 may receive, collect, log, or store particular data associated with the user for any purpose. A user may restrict her data at various levels. For example, in one level, the data may not be accessed by the computing server 130 for purposes other than displaying the data in the user's own profile. In another level, the user may authorize anonymization of her data and participate in studies and researches conducted by the computing server 130 such as a large scale genetic study. In yet another level, the user may turn some portions of her genealogical data public to allow the user to be discovered by other users (e.g., potential relatives) and be connected in one or more family trees.

The sample pre-processing engine 215 receives and pre-processes data received from various sources to change the data into a format used by the computing server 130. For genealogical data, the sample pre-processing engine 215 may receive data from an individual via the user interface 115 of the client device 110. To collect the user data (e.g., genealogical and survey data), the computing server 130 may cause an interactive user interface on the client device 110 to display interface elements in which users can provide genealogical data and survey data. Additional data may be obtained from scans of public records. The data may be manually provided or automatically extracted via, for example, optical character recognition (OCR) performed on census records, town or government records, or any other item of printed or online material. Some records may be obtained by digitalizing written records such as older census records, birth certificates, death certificates, etc.

The sample pre-processing engine 215 may also receive raw data from genetic data extraction service server 125. The genetic data extraction service server 125 may perform laboratory analysis of biological samples of users and generate sequencing results in the form of digital data. The sample pre-processing engine 215 may receive the raw genetic datasets from the genetic data extraction service server 125. The human genome mutation rate is estimated to be 1.1*10\hat{ }−8 per site per generation. This leads to a variant approximately every 300 base pairs. Most of the mutations that are passed down to descendants are related to single-nucleotide polymorphism (SNP). SNP is a substitution of a single nucleotide that occurs at a specific locus in the genome. The sample pre-processing engine 215 may convert the raw base pair sequence into a sequence of genotypes of target SNP loci. Alternatively, the pre-processing of this conversion may be performed by the genetic data extraction service server 125. The sample pre-processing engine 215 identifies autosomal SNPs in an individual's genetic dataset. For example, 700,000 autosomal SNPs may be identified in an individual's data and may be stored in genetic data store 205. Alternatively, in one embodiment, a genetic dataset may include at least 10,000 SNP loci. In another embodiment, a genetic dataset may include at least 100,000 SNP loci. In yet another embodiment, a genetic dataset may include at least 500,000 SNP loci. In yet another embodiment, a genetic dataset may include at least 1,000,000 SNP loci. The sample pre-processing engine 215 may also convert the nucleotides into bits. The identified SNPs, in bits or in other suitable formats, may be provided to the phasing engine 220 which phases the individual's diploid genotypes to generate a pair of haplotypes for each user. In some embodiments, the genotype data for an individual is statistically inferenced at one or more unobserved loci using known haplotypes, e.g., from a previously sequenced population.

The phasing engine 220 phases diploid genetic dataset into a pair of haploid genetic datasets. An individual's haplotype may refer to a collection of alleles (e.g., a sequence of alleles) that are inherited from a parent. In one context, a haplotype may also refer to a collection of alleles that corresponds to a genetic segment. In other contexts, a haplotype may refer to a specific allele at a SNP locus. For example, a sequence of haplotypes may refer to a sequence of alleles of an individual that are inherited from a parent.

Phasing may include a process of determining the assignment of alleles (particularly heterozygous alleles) to chromosomes. Owing to sequencing conditions and other constraints, a sequencing result often includes data regarding a pair of alleles at a given SNP locus of a pair of chromosomes but may not be able to distinguish which allele belongs to which specific chromosome. The phasing engine 220 uses a genotype phasing algorithm to assign one allele to a first chromosome and another allele to another chromosome. The genotype phasing algorithm may be developed based on an assumption of linkage disequilibrium (LD), which states that haplotype in the form of a sequence of alleles tends to cluster together. The phasing engine 220 is configured to generate phased sequences that are also commonly observed in many other samples. Put differently, haplotype sequences of different individuals tend to cluster together. A haplotype-cluster model may be generated to determine the probability distribution of a haplotype that includes a sequence of alleles. The haplotype-cluster model may be trained based on labeled data that includes known phased haplotypes from a trio (parents and a child). A trio is used as training sample because the correct phasing of the child is almost certain by comparing the child's genotypes to the parent's genetic datasets. The haplotype-cluster model may be generated iteratively along with the phasing process with a large number of unphased genotype datasets.

By way of example, the phasing engine 220 may use a directed acyclic graph model such as a hidden Markov model (HMM) to perform phasing of a target genotype dataset. The directed acyclic graph may include multiple levels, each level having multiple nodes representing different possibilities of haplotype clusters. An emission probability of a node, which may represent the probability of having a particular haplotype cluster given an observation of the genotypes may be determined based on the probability distribution of the haplotype-cluster model. A transition probability from one node to another may be initially assigned to a non-zero value and be adjusted as the directed acyclic graph model and the haplotype-cluster model are trained. Various paths are possible in traversing different levels of the directed acyclic graph model. The phasing engine 220 determines a statistically likely path, such as the most probable path or a probable path that is at least more likely than 95% of other possible paths, based on the transition probabilities and the emission probabilities. A suitable dynamic programming algorithm such as the Viterbi algorithm may be used to determine the path. The determined path may represent the phasing result. U.S. patent application Ser. No. 15/591,099, entitled “Haplotype Phasing Models,” filed on Oct. 19, 2015, describes one possible embodiment of haplotype phasing.

The IBD estimation engine 225 estimates the amount of shared genetic segments between a pair of individuals based on phased genotype data (e.g., haplotype datasets) that are stored in the genetic data store 205. IBD segments may be segments identified in a pair of individuals that are putatively determined to be inherited from a common ancestor. The IBD estimation engine 225 retrieves a pair of haplotype datasets for each individual. The IBD estimation engine 225 may divide each haplotype dataset sequence into a plurality of windows. Each window may include a fixed number of SNP loci (e.g., about 100 SNP loci). The IBD estimation engine 225 identifies one or more seed windows in which the alleles at all SNP loci in at least one of the phased haplotypes between two individuals are identical. The IBD estimation engine 225 may expand the match from the seed windows to nearby windows until the matched windows reach the end of a chromosome or until a homozygous mismatch is found, which indicate the mismatch is not attributable to potential errors in phasing. The IBD estimation engine 225 determines the total length of matched segments, which may also be referred to as IBD segments. The length may be measured in the genetic distance in the unit of centimorgans (cM). The computing server 130 may save data regarding individual pairs who share a length of IBD segments exceeding a predetermined threshold (e.g., 6 cM), such as in the genealogical data store 200. U.S. patent application Ser. No. 14/029,765, entitled “Identifying Ancestral Relationships Using a Continuous stream of Input,” filed on Sep. 17, 2013, and U.S. patent application Ser. No. 15/519,104, entitled “Reducing Error in Predicted Genetic Relationships,” filed on Apr. 13, 2017, describe example embodiments of IBD estimation.

Typically, individuals who are closely related share a relatively large number of IBD segments, and the IBD segments tend to have greater lengths (individually or in aggregate across one or more chromosomes). In contrast, individuals who are more distantly related share relatively fewer IBD segments, and these segments tend to be shorter (individually or in aggregate across one or more chromosomes). For example, while close family members often share upwards of 71 cM of IBD (e.g., third cousins), more distantly related individuals may share less than 12 cM of IBD. The extent of relatedness in terms of IBD segments between two individuals may be referred to as IBD affinity. For example, the IBD affinity may be measured in terms of the length of IBD segments between two individuals.

Community assignment engine 230 assigns individuals to one or more genetic communities. A genetic community may correspond to an ethnic origin or a group of people descended from a common ancestor. The granularity of genetic community classification may vary depending on embodiments and methods used in assigning communities. For example, in one embodiment, the communities may be African, Asian, European, etc. In another embodiment, the European community may be divided into Irish, German, Swedes, etc. In yet another embodiment, the Irish may be further divided into Irish in Ireland, Irish immigrated to America in 1800, Irish immigrated to America in 1900, etc. The community classification may also depend on whether a population is admixed or unadmixed. For an admixed population, the classification may further be divided based on different ethnic origins in a geographical region.

Community assignment engine 230 may assign individuals to one or more genetic communities based on their genetic datasets using machine learning models trained by unsupervised learning or supervised learning. In an unsupervised approach, the community assignment engine 230 may generate data representing a partially connected undirected graph. In this approach, the community assignment engine 230 represents individuals as nodes. Some nodes are connected by edges whose weights are based on IBD affinity between two individuals represented by the nodes. For example, if the total length of two individuals' shared IBD segments does not exceed a predetermined threshold, the nodes are not connected. The edges connecting two nodes are associated with weights that are measured based on the IBD affinities. The undirected graph may be referred to as an IBD network. The community assignment engine 230 uses clustering techniques such as modularity measurement (e.g., Louvain method) to classify nodes into different clusters in the IBD network. Each cluster may represent a community. The community assignment engine 230 may also determine sub-clusters, which represent sub-communities. The computing server 130 saves the data representing the IBD network and clusters in the IBD network data store 235. U.S. patent application Ser. No. 15/168,011, entitled “Discovering Population Structure from Patterns of Identity-By-Descent,” filed on May 28, 2016, describes one possible embodiment of community detection and assignment.

The community assignment engine 230 may also assign communities using supervised techniques. For example, genetic datasets of known genetic communities (e.g., individuals with confirmed ethnic origins, or individuals clustered together based on IBD commonalities) may be used as training sets that have labeled of the genetic communities. Supervised machine learning classifiers, such as logistic regressions, support vector machines, random forest classifiers, and neural networks may be trained using the training set with labels. A trained classifier may distinguish binary or multiple classes. For example, a binary classifier may be trained for each community of interest to determine whether a target individual's genetic dataset belongs or does not belong to the community of interest. A multi-class classifier such as a neural network may also be trained to determine whether the target individual's genetic dataset most likely belongs to one of several possible genetic communities.

Reference panel sample store 240 stores reference panel samples for different genetic communities. Some individuals' genetic data may be the most representative of a genetic community. Their genetic datasets may serve as reference panel samples. For example, some alleles of genes may be over-represented (e.g., being highly common) in a genetic community. Some genetic datasets include alleles that are commonly present among members of the community. Reference panel samples may be used to train various machine learning models in classifying whether a target genetic dataset belong to a community, in determining the ethnic composition of an individual, and in determining the accuracy in any genetic data analysis, such as by computing a posterior probability of a classification result from a classifier.

A reference panel sample may be identified in different ways. In one embodiment, an unsupervised approach in community detection may apply the clustering algorithm recursively for each identified cluster until the sub-clusters contain a number of nodes that is smaller than a threshold (e.g., contains fewer than 1000 nodes). For example, the community assignment engine 230 may construct a full IBD network that includes a set of individuals represented by nodes and generate communities using clustering techniques. The community assignment engine 230 may randomly sample a subset of nodes to generate a sampled IBD network. The community assignment engine 230 may recursively apply clustering techniques to generate communities in the sampled IBD network. The sampling and clustering may be repeated for different randomly generated sampled IBD networks for various runs. Nodes that are consistently assigned to the same genetic community when sampled in various runs may be classified as a reference panel sample. The community assignment engine 230 may measure the consistency in terms of a predetermined threshold. For example, if a node is classified to the same community 95% (or another suitable threshold) of times whenever the node is sampled, the genetic dataset corresponding to the individual represented by the node may be regarded as a reference panel sample. Additionally, or alternatively, the community assignment engine 230 may select N most consistently assigned nodes as a reference panel for the community.

Other ways to generate reference panel samples are also possible. For example, the computing server 130 may collect a set of samples and gradually filter and refine the samples until high-quality reference panel samples are selected.

The ethnicity estimation engine 245 estimates the ethnicity composition of a genetic dataset of a target individual. The genetic datasets used may be genotype datasets or haplotype datasets. For example, the ethnicity estimation engine 245 estimates the ancestral origins (e.g., ethnicity) based on the individual's genotypes or haplotypes at the SNP loci. To take a simple example of three ancestral populations corresponding to African, European and Native American, an admixed user may have nonzero estimated ethnicity proportions for all three ancestral populations, with an estimate such as [0.05, 0.65, 0.30], indicating that the user's genome is 5% attributable to African ancestry, 65% attributable to European ancestry and 30% attributable to Native American ancestry. The ethnicity estimation engine 245 generates the ethnic composition estimate and stores the estimated ethnicities in a data store of computing server 130 with a pointer in association with a particular user.

In one embodiment, the ethnicity estimation engine 245 divides a target genetic dataset into a plurality of windows (e.g., about 1000 windows). Each window includes a small number of SNP loci (e.g., 300 SNP loci). The ethnicity estimation engine 245 may use a directed acyclic graph model to determine the ethnic composition of the target genetic dataset. The directed acyclic graph may represent a trellis of an inter-window hidden Markov model (HMM). The graph includes a sequence of a plurality of levels. Each level, representing a window, include a plurality of nodes. The nodes representing different possibilities of labels of genetic communities (e.g., ethnicities) for the window. A node may be labeled with one or more ethnic labels. For example, a level includes a first node with a first label representing the likelihood that the window of SNP loci belongs to a first ethnicity and a second node with a second label representing the likelihood that the window of SNP loci belongs to a second ethnicity. Each level includes multiple nodes so that there are many possible paths to traverses the directed acyclic graph.

The nodes and edges in the directed acyclic graph may be associated with different emission probabilities and transition probabilities. An emission probability associated with a node represents the likelihood that the window belongs to the ethnicity labeling the node given the observation of SNP loci in the window. The ethnicity estimation engine 245 determines the emission probabilities by comparing SNP loci in the window corresponding to the target genetic dataset to corresponding SNP loci in the windows in various reference panel samples of different genetic communities stored in the reference panel sample store 240. The transition probability between two nodes represents the likelihood of transition from one node to another across two levels. The ethnicity estimation engine 245 determines a statistically likely path, such as the most probable path or a probable path that is at least more likely than 95% of other possible paths, based on the transition probabilities and the emission probabilities. A suitable dynamic programming algorithm such as the Viterbi algorithm or the forward-backward algorithm may be used to determine the path. After the path is determined, the ethnicity estimation engine 245 determines the ethnic composition of the target genetic dataset by determining the label compositions of the nodes that are included in the determined path. U.S. patent application Ser. No. 15/209,458, entitled “Local Genetic Ethnicity Determination System,” filed on Jul. 13, 2016, describes an example embodiment of ethnicity estimation.

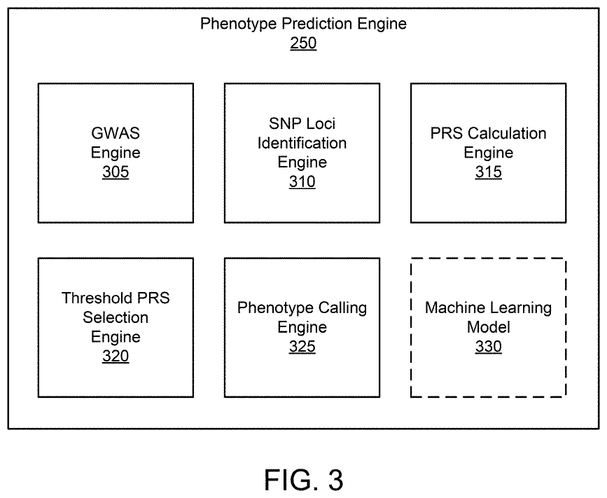

The phenotype prediction engine 250 predicts whether a target individual has a phenotypic trait using a polygenic risk score (PRS) calculated from genetic datasets of the target individual. A phenotypic trait is an observable physical trait of human individuals. While phenotypic traits are used as examples in this present application, the association studies and other related prediction analyses may also be used to predict other traits that might or might not be related to the phenotypes or genetics of an individuals, such as traits like environmental factors, preferences, experiences, and other suitable identifiable traits that may be obtained from survey responses. The genetic datasets used may be genotype datasets or haplotype datasets. The phenotype prediction engine 250 identifies a subset of SNP loci from the plurality of SNP loci genotyped from a DNA sample of the target individual, with each SNP locus in the subset of SNP loci selected based on predictive ability of the phenotypic trait. The phenotype prediction engine 250 calculates a PRS for the target individual based on genotypes at the subset of SNP loci. The PRS is compared against one or more PRS thresholds for positively predicting that the target individual has the phenotypic trait. The prediction of the phenotypic trait using the PRS will be described further in FIGS. 3-7.

The threshold PRS may be identified using a plurality of training individuals. Among the plurality of training individuals, some are reported to have the phenotypic trait and others are reported to not have the phenotypic trait. The reports of the phenotypic trait may be obtained from any suitable sources. For example, the reports may be obtained from the survey responses stored in the survey response store 210, from consented medical history or records of the training individuals, from input data of the training individuals provided to the computing server 130, etc. The phenotype prediction engine 250 calculates a PRS for each training individual based on each individual's genotypes at the subset of SNP loci. Sweeping through the domain of PRS, i.e., a range of candidate threshold PRS, for the subset of SNP loci, the phenotype prediction engine 250 can calculate a receiver operating characteristic (ROC) curve plotting a true positive rate to a false positive rate for predicting the phenotypic trait among the training individuals. The true positive rate may correspond to a percentage of training individuals reported to have the phenotypic trait that were accurately predicted to have the phenotypic trait. The false positive rate may correspond to a percentage of training individuals reported to not have the phenotypic trait that were inaccurately predicted to have the phenotypic trait. The phenotype prediction engine 250 selects one of the candidate threshold PRS as the threshold PRS based on the true positive rate and the false positive rate corresponding to the selected one of the candidate threshold PRS. The selected candidate threshold PRS may be selected to ensure a sufficiently high true positive rate and a sufficiently low false positive rate, i.e., an optimal threshold PRS that maximizes the true rate and minimizes the false positive rate. For example, ratios of the true positive rate to the false rate may be determined over different candidate threshold PRS. A threshold PRS corresponding to the highest value of the ratio may be selected. Other suitable ways may also be used to select the threshold PRS.

The front-end interface 255 may display various results determined by the computing server 130. The results and data may include the IBD affinity between a user and another individual, the community assignment of the user, the ethnicity estimation of the user, phenotype prediction and evaluation, genealogical data search, family tree and pedigree, relative profile and other information. The front-end interface 255 may be a graphical user interface (GUI) that displays various information and graphical elements. The front-end interface 255 may take different forms. In one case, the front-end interface 255 may be a software application that can be displayed at an electronic device such as a computer or a smartphone. The software application may be developed by the entity controlling the computing server 130 and be downloaded and installed at the client device 110. In another case, the front-end interface 255 may take the form of a webpage interface of the computing server 130 that allows users to access their family tree and genetic analysis results through web browsers. In yet another case, the front-end interface 255 may provide an application program interface (API).

Phenotype Prediction with Polygenic Risk Scoring

FIG. 3 is a block diagram of an architecture of an example phenotype prediction engine 250, in accordance with an embodiment. The phenotype prediction engine 250 predicts whether an individual has a particular phenotypic trait based on the individual's genetic dataset. The phenotype prediction engine 250 has a genome-wide association study (GWAS) engine 305, a SNP loci identification engine 310, a PRS calculation engine 315, a threshold PRS selection engine 320, and a phenotype calling engine 325. Alternatively, or additionally, the phenotype prediction engine 250 uses a machine learning model 330 for predicting the phenotypic trait. In various embodiments, the phenotype prediction engine 250 includes additional or fewer components than those listed herein. The phenotype prediction engine 250 may also include different components. Moreover, functions described under the engines may be variably distributed according to other implementations of the principles described herein.

In conducting a GWAS, the GWAS engine 305 obtains a plurality of training individuals. The plurality of training individuals may be narrowed according to an ethnicity prediction, e.g., the training individuals are all of European ethnicity. In another embodiment, the training individuals are rather (or additionally) narrowed by genetic community, providing a more granular scale than ethnicity. The determination of ethnicity and/or genetic community may be performed using the community assignment engine 230 and/or the ethnicity estimation engine 245. The training individuals include at least a first subset of positive training individuals, i.e., training individuals reported to have the phenotypic trait in consideration and a second subset of negative training individuals, i.e., training individuals reported to not have the phenotypic trait. Each training individual has a genetic dataset comprising genotypes over a plurality of SNP loci genotyped, e.g., by the computing server 130. Reporting by an individual on whether or not that individual has a phenotypic trait may be collected in surveys and stored, e.g., in the survey response store 210.

The GWAS engine 305 conducts a GWAS for a phenotypic trait with the plurality of training individuals. In conducting the GWAS, the GWAS engine 305, at each SNP locus genotyped in the genetic datasets of the training individuals, counts a first percentage of positive training individuals with a genotype including the SNP and counts a second percentage of negative training individuals with a genotype including the SNP. The GWAS engine 305 can calculate a predictive score for each SNP locus according to the first percentage and the second percentage, e.g., an odds ratio of the first percentage to the second percentage. In some embodiments, the GWAS engine 305 calculates a p-value score as the predictive score, wherein the p-value score indicates a likelihood of observing a result that is equal to or more extreme than the actual observed percentages. The GWAS engine 305 may tabulate the predictive scores (e.g., p-value scores) for each SNP locus.

In some embodiments, the phenotypic trait comprises a plurality of labels, e.g., many eye colors. In these embodiments, the GWAS engine 305 may conduct the GWAS by considering each label against the remaining labels. For example, when considering a first label, each training individual of that first label would be considered a positive training individual while the remaining training individuals would be considered a negative training individual. The GWAS engine 305 may then apply a similar principle described above to calculate a predictive score for each SNP loci according to percentages of the positive training individuals and the negative training individuals. The GWAS engine 305 may iterate through each of the labels calculating a predictive score for each SNP loci indicating predictive ability of each label. The predictive scores for a given SNP loci may be aggregated in some manner, e.g., average, median, maximum, minimum, etc.

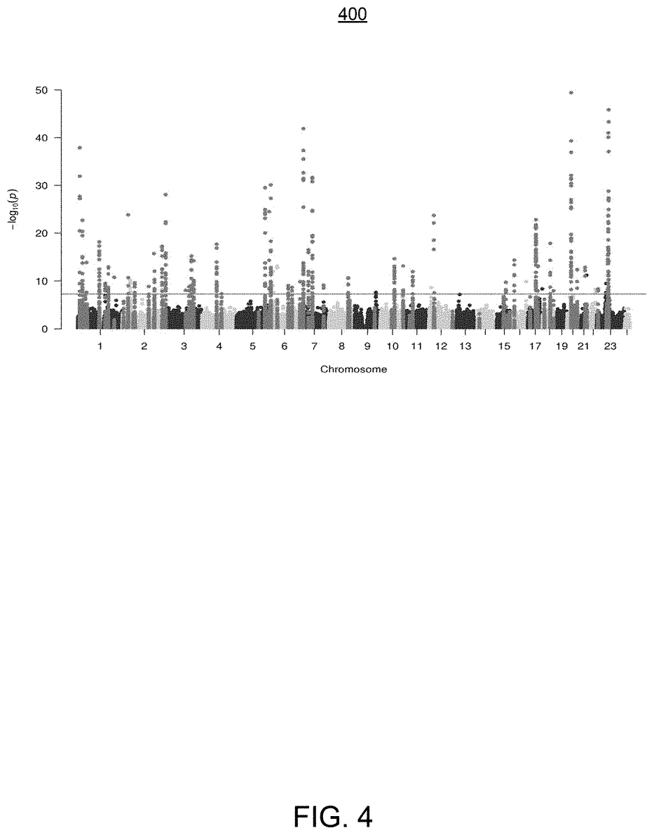

Referring to FIG. 4, FIG. 4 is an example graph 400 of SNP loci predictive of a phenotypic trait (e.g., male pattern baldness) in a cohort of individuals of European ethnicity, in accordance with an embodiment. This graph is a Manhattan plot with results of a GWAS conducted on the cohort of individuals of European ethnicity for the phenotypic trait of male pattern baldness. The x-axis truncates SNP loci distributed throughout the human genome by chromosomes. The y-axis is a -log(p-value) for each SNP locus, thus a large value on the y-axis represents a high predictive ability of a given SNP locus. Of note, there are peaks in the Manhattan plot corresponding to several SNP loci that are highly predictive of male pattern baldness.

Referring back to FIG. 3, the SNP loci identification engine 310 selects SNP loci for consideration in determining a PRS. The SNP loci identification engine 310 obtains the predictive scores (e.g., p-value scores) for each SNP locus genotyped by the computing server 130 for individuals. The SNP loci identification engine 310 identifies a subset of the SNP loci with a predictive score threshold. Considering the example with the predictive score calculated as a p-value score, the SNP loci identification engine 310 identifies the subset by including SNP loci with a p-value score below the p-value threshold, e.g., 0.05, 0.01, 1E-5, etc. In some embodiments, the SNP loci identification engine 310 further filters SNP loci that have some degree of linkage disequilibrium with other SNP loci. Linkage disequilibrium (LD) refers to the non-random association between alleles at different loci in the human population. To measure a degree of LD between two SNPs at two SNP loci, a difference is calculated between a product of the likelihoods of observing the SNPs independently and the likelihood of observing the SNPs together. The SNP loci identification engine 310 may set a threshold for filtering SNPs with a degree of LD above the threshold. The threshold may be set for greedy or non-greedy inclusion of SNP loci in the subset of SNP loci.

The PRS calculation engine 315 calculates a PRS for an individual according to the individual's genotypes for the identified subset of SNP loci. The PRS calculation engine 315 maintains a PRS function that inputs the genotypes over the identifies subset of SNP loci and outputs a PRS. In some implementations, the PRS function is a linear function with a weight for each SNP locus in the identified subset. Genotypes of the SNP locus may be binary values (e.g., whether or not the individual has the dominant allele) or trinary values (e.g., homozygous with the first allele, heterozygous, homozygous with the second allele). The weights may be determined based on predictive scores from the GWAS engine 305. The PRS function may further normalize the PRS, e.g., limiting the domain of the PRS in the range of [0, 1]. In other embodiments, the PRS calculation engine 315 may employ other techniques in calculating the PRS, e.g., Bayesian approaches considering linkage disequilibrium prediction, penalized regression techniques, etc.

The threshold PRS selection engine 320 selects a threshold PRS for predicting the phenotypic trait. The threshold PRS selection engine 320 takes a plurality of training individuals (which can be the same plurality used to conduct the GWAS by the GWAS engine 305). The training individuals are collected according to similar principles as described above in the GWAS engine 305. The threshold PRS selection engine 320 obtains a PRS score for each of the training individuals calculated by the PRS calculation engine 315 according to an identified subset of SNP loci with a threshold predictive score (e.g., SNP loci with p-value scores under 0.05). The threshold PRS selection engine 320 sweeps through a domain of the PRS for the identified subset, i.e., a range of candidate threshold PRS (e.g., the domain range from [0, 1]), to test threshold PRSs and predicts a number of the training individuals with PRSs above the test threshold PRS to have the phenotypic trait. The threshold PRS calculation engine 320 can calculate a true positive rate as a percentage of positive training individuals, training individuals reported to have the phenotypic trait, that were accurately predicted to have the phenotypic trait. The threshold PRS calculation engine 320 can also calculate a false positive rate as a percentage of negative training individuals, training individuals reported to not have the phenotypic trait, that were inaccurately predicted to have the phenotypic trait. The threshold PRS calculation engine 320 may perform additional statistical calculations on the prediction, e.g., true negative, false negative, etc.

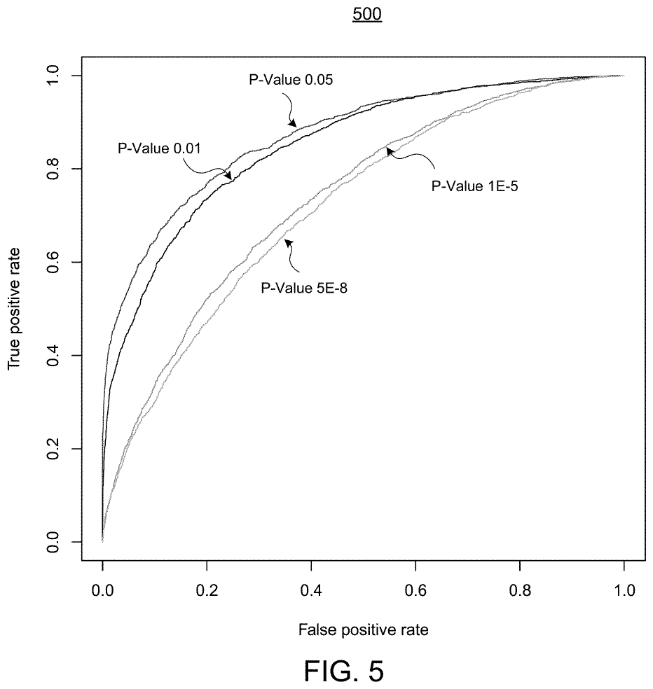

According to one or more embodiments, the threshold PRS selection engine 320 selects one of the candidate threshold PRS over the range of candidate threshold PRS as the threshold PRS based on at least the true positive rate and the false positive rate corresponding to the selected candidate threshold PRS. The selected candidate threshold PRS may be selected as to achieve a sufficiently high true positive rate and a sufficiently low false positive rate. In an ideal case, a perfect threshold PRS has a true positive rate in predicting positive training individuals as 100% and a false positive rate as 0%. Thus, the threshold PRS that has a sufficiently high true positive rate and a sufficiently low false positive rate is closest to the perfect threshold PRS, illustrated below in FIG. 5. In other embodiments, the threshold PRS is selected based on other statistics calculated (e.g., true negative, false negative etc.) in substitution of or in addition to the true positive and false positive rates.

In embodiments with multiple labels, the threshold PRS selection engine 320 may select a PRS threshold for each label of the phenotypic trait to stratify the prediction. For each label, the threshold PRS selection engine 320 may (similarly with the binary label) sweep through the domain of the PRS to test threshold PRSs to generate a ROC curve plotting true positive rate against false positive rate, wherein the training individuals of the label being considered would be considered positive training individuals while remaining training individuals are considered negative training individuals. The PRS selection engine 320 may select the optimal threshold PRS from the ROC curve as the PRS threshold for that label. Upon selecting a PRS threshold for each label, the domain of the PRS may be stratified with the PRS thresholds such that a range of PRS scores would yield a prediction for each label. In an example with three labels, the threshold PRS selection engine 320 chooses a PRS threshold of 0.2 for the first label, a PRS threshold of 0.6 for the second label, and a PRS threshold of 0.8 for the third label. Such that a PRS within [0, 0.2) calls a null label (e.g., none of the three labels), a PRS within [0.2, 0.6) calls the first label, a PRS within [0.6, 0.8) calls the second label, and a PRS within [0.8, 1.0] calls the third label.

Referring to FIG. 5, FIG. 5 is a graph 500 of predictive sensitivity over a domain of polygenic risk score for four different subsets of SNP loci, in accordance with an embodiment. There are four different subsets of SNP loci corresponding to different p-value score cutoffs. A first subset with a p-value cutoff of 0.05 includes roughly 30,000 SNP loci used for calculating the PRS. A second subset with a p-value cutoff of 0.01 includes roughly 8,000 SNP loci. A third subset with a p-value cutoff of 1E-5 includes roughly 400 SNP loci. A fourth subset with a p-value cutoff of 5E-8 includes roughly 200 SNP loci. For each subset of SNP loci, the threshold PRS calculation engine 320 sweeps through the domain of PRS and calculates a true positive rate and a false positive rate.

The graph 500 plots ROC curves for the four subsets over the domain of PRS. The ideal threshold PRS is at (1, 0) where the true positive rate is 100% and the false positive rate is 0%. The threshold PRS can be identified from each subset as the closest (e.g., by Euclidean distance) to the point (1, 0), i.e., sufficiently high true positive rate and sufficiently low false positive rate. For example, with the ROC curve for the p-value of 0.05, the closest point to the ideal threshold PRS is roughly (0.77, 0.21), e.g., which might correspond to a threshold PRS of 0.4. The threshold PRS selection engine 320 can identify that PRS of 0.4 as the threshold PRS for use in predicting the phenotypic trait.

In some embodiments, the SNP loci identification engine 310 determines a p-value threshold that optimizes overall predictability of the subset of SNP loci. As shown in the graph 500, each subset's ROC curve is unique with varying degrees of overall predictability. ROC curves that tend towards the ideal threshold PRS have better overall predictability. As exampled in the graph 500, the subsets can be ordered according to overall predictability, with the following order: the first subset, the second subset, the third subset, and the fourth subset. The overall predictability may be based on the closest PRS on the ROC curve to the ideal threshold PRS or some holistic measure, which ROC curve is greater over a majority of the false positive domain.

The phenotype calling engine 325 predicts whether an individual has a phenotypic trait according to the individual's PRS. The phenotype calling engine 325 obtains a PRS for an individual calculated by the PRS calculation engine 315 according to an identified subset of SNP loci (e.g., selected with a p-value cutoff of 0.05). The phenotype calling engine 325 further obtains the threshold PRS (or in cases with multiple labels for phenotypic trait, the PRS thresholds) for predicting whether an individual has the particular phenotypic trait from the threshold PRS selection engine 320. The phenotype calling engine 325 compares the individual's PRS to the PRS threshold(s) and predicts whether the individual likely has the phenotypic trait based on the comparison (or predicts one of the labels of the phenotypic trait in pluralistic phenotypic traits). As a numerical example, the individual's PRS may be 0.5 which, when compared against the threshold PRS of 0.4, results in a prediction that the individual has the phenotypic trait. The phenotype calling engine 325 may provide the prediction—likely has or likely does not have the phenotypic trait—to the front-end interface 255, e.g., for presentation to the individual or to other users via a client device 110. In embodiments where the phenotypic trait is a continuum, the phenotype calling engine 325 may retrieve a trained regression that regresses the phenotypic trait against the PRS. The phenotype calling engine 325 inputs the individual's PRS into the trained regression to determine a value on the continuum for the phenotypic trait.

In some embodiments, the phenotype calling engine 325 may alternatively or additionally implement a machine learning model 330 to predict the phenotypic trait. The machine learning model 330 inputs a feature vector comprising genotypes over an identified subset of SNP loci, e.g., identified by the SNP loci identification engine 310. Some or all of the identified subset of SNP loci may also be identified via other techniques. The machine learning model 330 outputs a prediction of a likelihood the individual likely has the phenotype. The prediction may be a binary prediction of whether the individual has or does not have the phenotype, e.g., binary classification, or may be a percent likelihood that the individual has the phenotype. In embodiments where the phenotypic trait comprises a plurality of labels, the prediction may be a multiclass prediction of which label the individual likely has or a percent likelihood that the individual has each of the labels in the phenotypic trait considered. For example, the phenotypic trait considered is eye color with multiple labels such as blue, green, hazel, or brown, wherein the prediction could be the most likely eye color or a percentage likelihood associated with each eye color. In yet other embodiments, the phenotypic trait may be a continuum (e.g., height) wherein a regression is generated that outputs a prediction on the continuum. The machine learning model may be trained as the regression taking feature vectors and outputting the phenotypic trait. The phenotype prediction engine 250 may also train the machine learning model 330 with the plurality of training individuals. Different machine learning techniques may be implemented such as a support vector machine (SVM), boosting for other algorithms (e.g., AdaBoost), neural networks, logistic regression, naïve Bayes, memory-based learning, random forests, bagged trees, decision trees, boosted trees, or boosted stumps. The machine learning model may consider other features in substitution or in addition to the SNP loci. These features include but are not limited to PRS, age, sex, array sequenced on, other survey questions, phenotypes of ancestors or relatives, ethnicity, community.

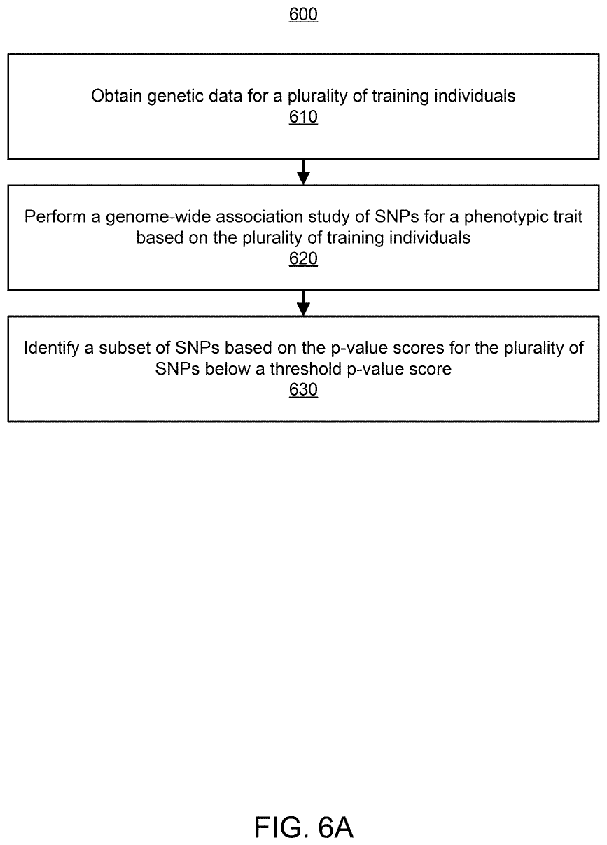

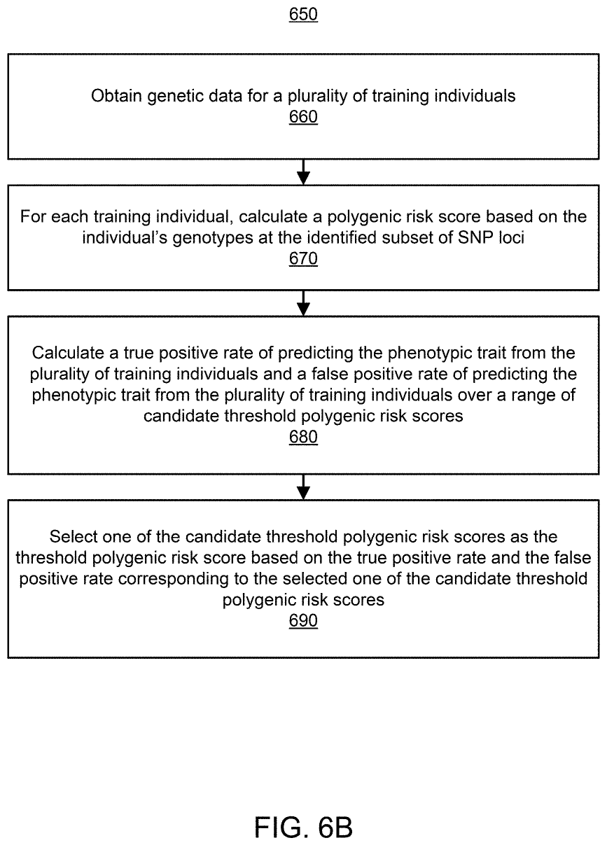

FIG. 6A is a flowchart illustrating a process 600 of identifying a subset of SNP loci for consideration in predicting a phenotypic trait, in accordance with an embodiment. The process 600 may be performed by the phenotype prediction engine 250 or more generally by another computer system. In other embodiments, the process 600 includes additional or fewer steps than those listed in FIG. 6A. The steps may also vary in order in other implementations.

At step 610, the phenotype prediction engine 250 obtains genetic data for a plurality of training individuals. The genetic data includes a plurality of reads of SNPs that describe an individual's genotype at each of a plurality of SNP loci. The plurality of training individuals includes positive training individuals reported to have the phenotypic trait and negative training individuals reported to not have the phenotypic trait. The plurality of training individuals may be further screened such that all the training individuals have one or more common characteristics, which may be determined by the computing server 130. Examples of common characteristics may include sex, ethnicity, genetic community, age, etc.

At step 620, the phenotype prediction engine 250 performs a GWAS of SNPs for a phenotypic trait based on the plurality of training individuals. In conducting the GWAS, at each SNP locus genotyped in the genetic datasets of the training individuals, the phenotype prediction engine 250 counts a first percentage of positive training individuals with a genotype including the SNP and counts a second percentage of negative training individuals with a genotype including the SNP. A predictive score is calculated for each SNP locus according to the first percentage and the second percentage, e.g., an odds ratio of the first percentage to the second percentage. In primary embodiments, the predictive score is defined as a p-value score which indicates a likelihood of observing equal to or more extreme than the actual observed percentages.

At step 630, the phenotype prediction engine 250 identifies a subset of SNP loci based on the p-value scores for the plurality of SNP loci below a threshold p-value score. The identified subset of SNP loci may be used in calculating the PRS for an individual.

FIG. 6B is a flowchart illustrating a process 650 of identifying a threshold polygenic risk score for determining whether an individual has a phenotypic trait, in accordance with an embodiment. The process 650 may be performed by the phenotype prediction engine 250 or more generally by another computer system. In other embodiments, the process 650 includes additional or fewer steps than those listed in FIG. 6B. The steps may also vary in order in other implementations.

At step 660, the phenotype prediction engine 250 obtains genetic data for a plurality of training individuals. This is similar with step 610 in the process 600 of FIG. 6A.

At step 670, the phenotype prediction engine 250, for each training individual, calculates a polygenic risk score based on the individual's genotypes at the identified subset of SNP loci. The phenotype prediction engine 250 uses a PRS function that inputs the genotypes over the identified subset of SNP loci and outputs a PRS. In some implementations, the PRS function is a linear function with a weight for each SNP locus in the identified subset. The PRS function may further normalize the PRS, e.g., limiting the range of candidate threshold PRS in the range of [0, 1].

At step 680, the phenotype prediction engine 250 calculates a true positive rate of predicting the phenotypic trait from the plurality of training individuals and a false positive rate of predicting the phenotypic trait from the plurality of training individuals over a range of candidate threshold polygenic risk scores. The phenotype prediction engine 250 sweeps through a range of candidate threshold PRS for the identified subset (e.g., the domain ranges from [0, 1]) to test threshold PRSs and predicts a number of the training individuals with PRSs above the test threshold PRS to have the phenotypic trait. The threshold PRS calculation engine 320 can calculate a true positive rate as a percentage of positive training individuals, training individuals reported to have the phenotypic trait, that were accurately predicted to have the phenotypic trait. The threshold PRS calculation engine 320 can also calculate a false positive rate as a percentage of negative training individuals, training individuals reported to not have the phenotypic trait, that were inaccurately predicted to have the phenotypic trait.

At step 690, the phenotype prediction engine 250 selects one of the candidate threshold polygenic risk score as the threshold polygenic risk score based on the true positive rate and the false positive rate corresponding to the selected one of the candidate threshold polygenic risk scores. In some embodiments, the selected candidate threshold polygenic risk score is selected for having a sufficiently high true positive rate and a sufficiently low false positive rate that is closest to the perfect threshold PRS.

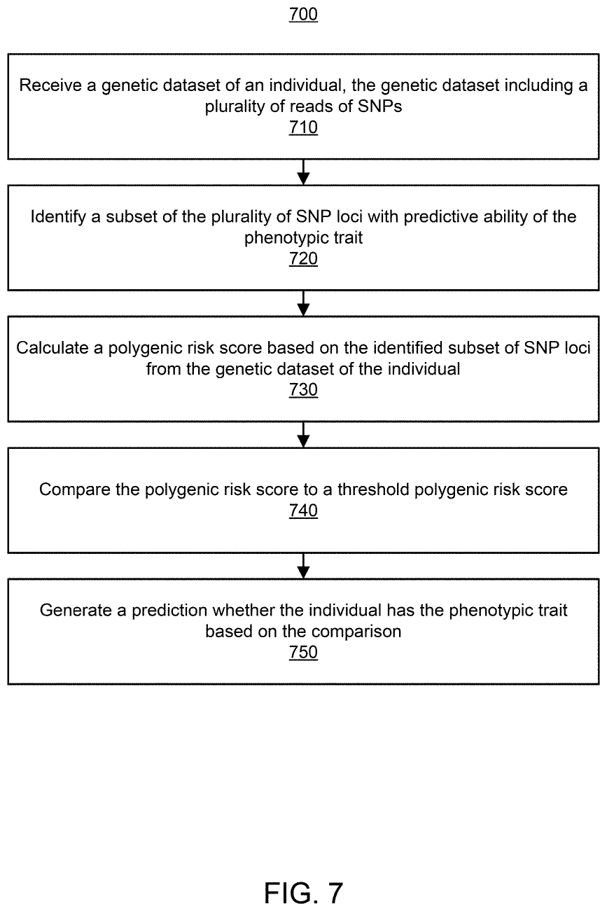

FIG. 7 is a flowchart illustrating a process 700 of predicting whether an individual has a phenotypic trait using PRS, in accordance with an embodiment. The process 700 may be performed by the phenotype prediction engine 250 or more generally by another computer system. In other embodiments, the process 700 includes additional or fewer steps than those listed in FIG. 7. The steps may also vary in order in other implementations.

At step 710, the phenotype prediction engine 250 receives a genetic dataset for an individual, the genetic dataset including a plurality of reads of SNPs. The genetic dataset includes genotypes for a plurality of SNP loci.

At step 720, the phenotype prediction engine 250 identifies a subset of the plurality of SNP loci with predictive ability of the phenotypic trait. This step 720 may correspond to the process 600 described in FIG. 6A.

At step 730, the phenotype prediction engine 250 calculates a PRS for the individual based on the identified subset of SNP loci from the genetic dataset of the individual. With the identified SNP loci from step 720, the phenotype prediction engine 250 calculates a PRS with a PRS function. The PRS function inputs the genotypes over the identified subset of SNP loci and outputs a PRS. In some implementations, the PRS function is a linear function with a weight for each SNP locus in the identified subset. The PRS function may further normalize the PRS, e.g., limiting the range of candidate threshold PRS in the range of [0, 1].

At step 740, the phenotype prediction engine 250 compares the PRS for the individual to a threshold PRS. The comparison may simply be determining whether the PRS for the individual is less than the threshold PRS or greater than or equal to the threshold PRS>

At step 750, the phenotype prediction engine 250 generates a prediction whether the individual has the phenotypic trait based on the comparison. The phenotype prediction engine 250 predicts that the individual likely has the phenotypic trait if the individual's PRS is greater than or equal to the threshold PRS. Alternatively, the phenotype prediction engine 250 predicts that the individual likely does not have the phenotypic trait if the individual's PRS is less than the threshold PRS. The phenotype prediction engine 250 may store the prediction, e.g., in the computing server 130. The phenotype prediction engine 250 may also present the prediction to the individual, e.g., via a client device.

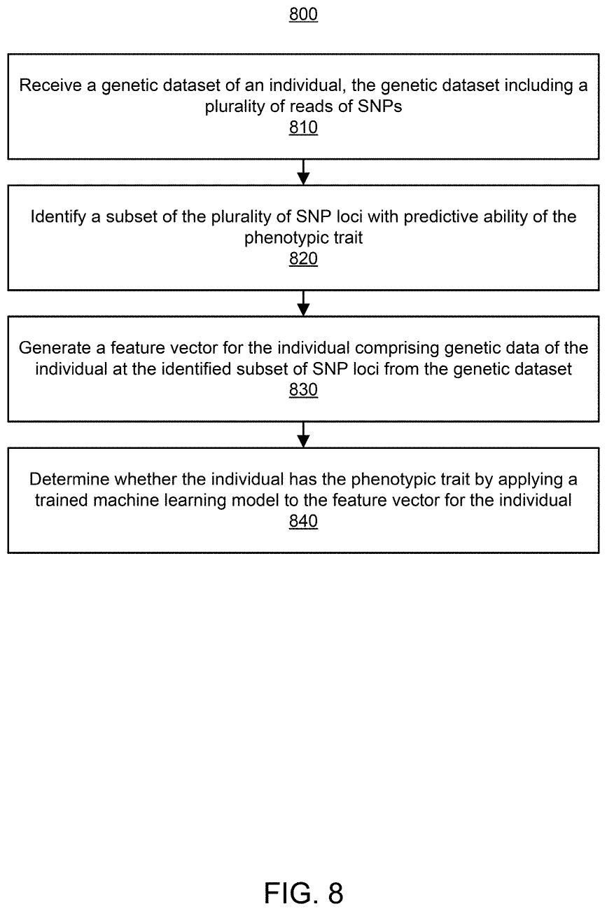

FIG. 8 is a flowchart illustrating a process 800 of predicting whether an individual has a phenotypic trait using a machine learning model, in accordance with an embodiment. The process 800 may be performed by the phenotype prediction engine 250 or more generally by another computer system. In other embodiments, the process 800 includes additional or fewer steps than those listed in FIG. 8. The steps may also vary in order in other implementations.

At step 810, the phenotype prediction engine 250 receives a genetic dataset for an individual, the genetic dataset including a plurality of reads of SNPs. The genetic dataset includes genotypes for a plurality of SNP loci.

At step 820, the phenotype prediction engine 250 identifies a subset of the plurality of SNP loci with predictive ability of the phenotypic trait. This step 820 may correspond to the process 600 described in FIG. 6A.

At step 830, the phenotype prediction engine 250 generates a feature vector for the individual comprising genetic data of the individual at the identified subset of SNP loci from the genetic dataset. The feature vector may also include other features including but not limited to age, sex, ethnicity, genetic community, phenotype of ancestors or relatives, other genetic data, other survey data, which may be used in substitution or in tandem with the genetic data at the identified subset of SNP loci.

At step 840, the phenotype prediction engine 250 determines whether the individual has the phenotypic trait by applying a machine learning model to the feature vector for the individual. The machine learning model is trained with training individuals reported to have or not to have the phenotypic trait (in a binary prediction). The machine learning model obtains genetic datasets (or other relevant data) to generate feature vectors for each of the training individuals. The feature vectors for the training individuals include at least the genetic data for each training individual at the identified subset of SNP loci. The machine learning model is trained with the feature vectors of the training individuals. In some embodiments, the machine learning model may be trained as a classifier (binary or multiclass) or as a regression. As a binary classifier, the machine learning model may output a prediction of whether the individual has or does not have the phenotypic trait, or a predicted likelihood that the individual has the phenotypic trait. As a multiclass classifier, the machine learning model may output a prediction of which label the individual has for the phenotypic trait (e.g., blue yes, or some degree of balding, etc.), or a likelihood that the individual is of each label. As a regression, the machine learning model may output a prediction on the continuum for a phenotypic trait (e.g., six feet tall, degree of freckles, etc.).

### Computing Machine Architecture

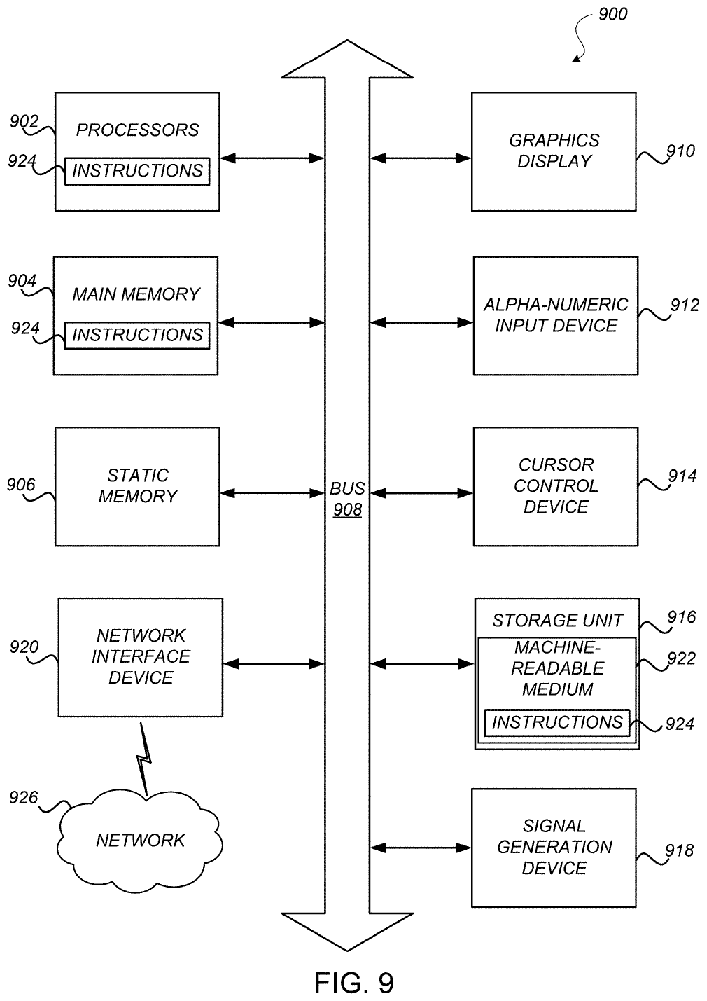

FIG. 9 is a block diagram illustrating components of an example computing machine that is capable of reading instructions from a computer-readable medium and execute them in a processor (or controller). A computer described herein may include a single computing machine shown in FIG. 9, a virtual machine, a distributed computing system that includes multiples nodes of computing machines shown in FIG. 9, or any other suitable arrangement of computing devices.

By way of example, FIG. 9 shows a diagrammatic representation of a computing machine in the example form of a computer system 900 within which instructions 924 (e.g., software, program code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 9 may correspond to any software, hardware, or combined components shown in FIGS. 1-3, including but not limited to, the client device 110, the computing server 130, and various engines, interfaces, terminals, and machines shown in FIG. 2. While FIG. 9 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 924 that specify actions to be taken by that machine. Further, while only a single machine is illustrated, the term “machine” and “computer” may also be taken to include any collection of machines that individually or jointly execute instructions 924 to perform any one or more of the methodologies discussed herein.

The example computer system 900 includes one or more processors 902 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 900 may also include a memory 904 that store computer code including instructions 924 that may cause the processors 902 to perform certain actions when the instructions are executed, directly or indirectly by the processors 902. Instructions can be any directions, commands, or orders that may be stored in different forms, such as equipment-readable instructions, programming instructions including source code, and other communication signals and orders. Instructions may be used in a general sense and are not limited to machine-readable codes.

One and more methods described herein improve the operation speed of the processors 902 and reduces the space required for the memory 904. For example, the machine learning methods described herein reduces the complexity of the computation of the processors 902 by applying one or more novel techniques that simplify the steps in training, reaching convergence, and generating results of the processors 902. The algorithms described herein also reduces the size of the models and datasets to reduce the storage space requirement for memory 904.

The performance of certain of the operations may be distributed among the more than processors, not only residing within a single machine, but deployed across a number of machines. In some example embodiments, the one or more processors or processor-implemented engines may be located in a single geographic location (e.g., within a home environment, an office environment, or a server farm). In other example embodiments, the one or more processors or processor-implemented engines may be distributed across a number of geographic locations. Even though in the specification or the claims may refer some processes to be performed by a processor, this should be construed to include a joint operation of multiple distributed processors.

The computer system 900 may include a main memory 904, and a static memory 906, which are configured to communicate with each other via a bus 908. The computer system 900 may further include a graphics display unit 910 (e.g., a plasma display panel (PDP), a liquid crystal display (LCD), a projector, or a cathode ray tube (CRT)). The graphics display unit 910, controlled by the processors 902, displays a graphical user interface (GUI) to display one or more results and data generated by the processes described herein. The computer system 900 may also include alphanumeric input device 912 (e.g., a keyboard), a cursor control device 914 (e.g., a mouse, a trackball, a joystick, a motion sensor, or other pointing instrument), a storage unit 916 (a hard drive, a solid state drive, a hybrid drive, a memory disk, etc.), a signal generation device 918 (e.g., a speaker), and a network interface device 920, which also are configured to communicate via the bus 908.

The storage unit 916 includes a computer-readable medium 922 on which is stored instructions 924 embodying any one or more of the methodologies or functions described herein. The instructions 924 may also reside, completely or at least partially, within the main memory 904 or within the processor 902 (e.g., within a processor's cache memory) during execution thereof by the computer system 900, the main memory 904 and the processor 902 also constituting computer-readable media. The instructions 924 may be transmitted or received over a network 926 via the network interface device 920.

While computer-readable medium 922 is shown in an example embodiment to be a single medium, the term “computer-readable medium” should be taken to include a single medium or multiple media (e.g., a centralized or distributed database, or associated caches and servers) able to store instructions (e.g., instructions 924). The computer-readable medium may include any medium that is capable of storing instructions (e.g., instructions 924) for execution by the processors (e.g., processors 902) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g. method, can be claimed in another claim category, e.g. computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof

Any of the steps, operations, or processes described herein may be performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

The following applications are incorporated by reference in their entirety for all purposes: (1) U.S. patent application Ser. No. 15/591,099, entitled “Haplotype Phasing Models,” filed on Oct. 19, 2015, (2) U.S. patent application Ser. No. 15/168,011, entitled “Discovering Population Structure from Patterns of Identity-By-Descent,” filed on May 28, 2016, (3) U.S. “Reducing Error in Predicted Genetic Relationships,” filed on Apr. 13, 2017, (4) U.S. patent application Ser. No. 15/209,458, entitled “Local Genetic Ethnicity Determination System,” filed on Jul. 13, 2016, and (5) U.S. patent application Ser. No. 14/029,765, entitled “Identifying Ancestral Relationships Using a Continuous stream of Input,” filed on Sep. 17, 2013.

What is claimed is:

1. A method for predicting a phenotypic trait for an individual, the method comprising:
receiving a genetic dataset of the individual, the genetic dataset including a plurality of reads at a plurality of single nucleotide polymorphism (SNP) loci;
identifying a subset of the plurality of SNP loci with predictive ability of the phenotypic trait;
calculating a polygenic risk score for the individual based on the genetic dataset of the individual at the identified subset of SNP loci;
comparing the polygenic risk score to a threshold polygenic risk score, the threshold polygenic risk score determined by:
obtaining genetic datasets for a plurality of training individuals,
calculating, for each training individual of the plurality of training individuals, a polygenic risk score for the training individual based on the genetic dataset of the training individual at the identified subset of SNP loci,
calculating a plurality of true positive rates of predicting the phenotypic trait of the plurality of training individuals over a range of candidate threshold polygenic risk scores, each true positive rate corresponding to one of the candidate threshold polygenic risk scores,
calculating a plurality of false positive rates of predicting the phenotypic trait of the plurality of training individuals over the range of candidate threshold polygenic risk scores, each false positive rate corresponding to one of the candidate threshold polygenic risk scores, and
selecting one of the candidate threshold polygenic risk scores as the threshold polygenic risk score based on the true positive rate and the false positive rate corresponding to the selected one of the candidate threshold polygenic risk scores; and
generating a prediction whether the individual has the phenotypic trait based on the comparison.

2. The method of claim 1, wherein the subset of SNP loci with the predictive ability of the phenotypic trait is identified with a genome-wide association study (GWAS) with the plurality of training individuals over the plurality of SNP loci, the GWAS comprising:
calculating a p-value score for each SNP locus based on a positive count of training individuals reported to have the phenotypic trait and a negative count of training individuals reported to not have the phenotypic trait; and
identifying the subset of SNP loci based on the p-value scores for the plurality of SNP loci being below a threshold p-value score;

3. The method of claim 2, wherein calculating the polygenic risk score comprises:
calculating a weight for each SNP locus of the subset of SNP loci based on the p-value score for the SNP locus; and
calculating the polygenic risk score by summing over each product of a genotype of the individual at a SNP locus in the subset of SNP loci and a corresponding weight for the SNP locus.

4. The method of claim 1, further comprising:
determining an ethnicity of the individual based on the genetic dataset;
wherein the plurality of training individuals is of the same ethnicity.

5. The method of claim 1, wherein the true positive rate for one of the candidate threshold polygenic risk scores is a percentage of the training individuals reported to have the phenotypic trait who are predicted to have the phenotypic trait based on the polygenic risk scores of the training individuals compared to the one of the candidate threshold polygenic risk scores.

6. The method of claim 1, wherein the false positive rate for one of the candidate threshold polygenic risk scores is a percentage of training individuals reported to not have the phenotypic trait who are predicted to have the phenotypic trait based on the polygenic risk scores of the training individuals compared to the one of the candidate threshold polygenic risk scores.

7. The method of claim 1, wherein generating a prediction whether the individual has the phenotypic trait based on the comparison comprises:
in response to the polygenic risk score of the individual being greater than or equal to the threshold polygenic risk score, determining that the individual likely has the phenotypic trait; and
in response to the polygenic risk score of the individual being less than the threshold polygenic risk score, determining that the individual likely does not have the phenotypic trait.

8. The method of claim 1, further comprising:
transmitting the prediction to a client device for displaying the prediction.

9. The method of claim 1, wherein the plurality of training individuals belongs to a genetic community, the genetic community determined based on identity-by-descent (IBD) affinities among the training individuals.

10. A non-transitory computer-readable storage medium storing instructions for predicting a phenotypic trait for an individual, the instructions, when executed by one or more processors, cause the one or more processors to perform operations comprising:
receiving a genetic dataset of the individual, the genetic dataset including a plurality of reads at a plurality of single nucleotide polymorphism (SNP) loci;
identifying a subset of the plurality of SNP loci with predictive ability of the phenotypic trait;
calculating a polygenic risk score for the individual based on the genetic dataset of the individual at the identified subset of SNP loci;
comparing the polygenic risk score to a threshold polygenic risk score, the threshold polygenic risk score determined by:
obtaining genetic datasets for a plurality of training individuals,
calculating, for each training individual of the plurality of training individuals, a polygenic risk score for the training individual based on the genetic dataset of the training individual at the identified subset of SNP loci,
calculating a plurality of true positive rates of predicting the phenotypic trait of the plurality of training individuals over a range of candidate threshold polygenic risk scores, each true positive rate corresponding to one of the candidate threshold polygenic risk scores,
calculating a plurality of false positive rates of predicting the phenotypic trait of the plurality of training individuals over the range of candidate threshold polygenic risk scores, each false positive rate corresponding to one of the candidate threshold polygenic risk scores, and
selecting one of the candidate threshold polygenic risk scores as the threshold polygenic risk score based on the true positive rate and the false positive rate corresponding to the selected one of the candidate threshold polygenic risk scores; and
generating a prediction whether the individual has the phenotypic trait based on the comparison.

11. The non-transitory computer-readable storage medium of claim 10, wherein the subset of SNP loci with the predictive ability of the phenotypic trait is identified with a genome-wide association study (GWAS) with the plurality of training individuals over the plurality of SNP loci, the GWAS comprising:
calculating a p-value score for each SNP locus based on a positive count of training individuals reported to have the phenotypic trait and a negative count of training individuals reported to not have the phenotypic trait; and
identifying the subset of SNP loci based on the p-value scores for the plurality of SNP loci being below a threshold p-value score;

12. The non-transitory computer-readable storage medium of claim 11, wherein calculating the polygenic risk score comprises:
calculating a weight for each SNP locus of the subset of SNP loci based on the p-value score for the SNP locus; and
calculating the polygenic risk score by summing over each product of a genotype of the individual at a SNP locus in the subset of SNP loci and a corresponding weight for the SNP locus.

13. The non-transitory computer-readable storage medium of claim 10, wherein the operations further comprise:
determining an ethnicity of the individual based on the genetic dataset;
wherein the plurality of training individuals is of the same ethnicity.

14. The non-transitory computer-readable storage medium of claim 10, wherein the true positive rate for one of the candidate threshold polygenic risk scores is a percentage of the training individuals reported to have the phenotypic trait who are predicted to have the phenotypic trait based on the polygenic risk scores of the training individuals compared to the one of the candidate threshold polygenic risk scores.

15. The non-transitory computer-readable storage medium of claim 10, wherein the false positive rate for one of the candidate threshold polygenic risk scores is a percentage of the training individuals reported to not have the phenotypic trait who are predicted to have the phenotypic trait based on the polygenic risk scores of the training individuals compared to the one of the candidate threshold polygenic risk scores.

16. The non-transitory computer-readable storage medium of claim 10, wherein generating a prediction whether the individual has the phenotypic trait based on the comparison comprises:
in response to the polygenic risk score of the individual being greater than or equal to the threshold polygenic risk score, determining that the individual likely has the phenotypic trait; and
in response to the polygenic risk score of the individual being less than the threshold polygenic risk score, determining that the individual likely does not have the phenotypic trait.

17. The non-transitory computer-readable storage medium of claim 10, wherein the operations further comprise:
transmitting the prediction to a client device for displaying the prediction.

18. The non-transitory computer-readable storage medium of claim 10, wherein the plurality of training individuals belongs to a genetic community, the genetic community determined based on identity-by-descent (IBD) affinities among the training individuals.

19. A method for predicting a phenotypic trait for an individual, the method comprising:
receiving a genetic dataset of the individual, the genetic dataset including a plurality of reads at a plurality of single nucleotide polymorphism (SNP) loci;
identifying a subset of the plurality of SNP loci with predictive ability of the phenotypic trait;
generating a feature vector for the individual comprising genetic data of the individual at the identified subset of SNP loci from the genetic dataset; and
determining whether the individual has the phenotypic trait by applying a trained machine learning model to the feature vector for the individual, the machine learning model trained by:
obtaining genetic datasets for a plurality of training individuals, a first subset of the training individuals reported to have the phenotypic trait and a second subset of training individuals reported to not have the phenotypic trait,
generating, for each training individual of the plurality of training individuals, a training feature vector comprising genetic data of the training individual at the identified subset of SNP loci from the genetic dataset, and
training the machine learning model with the plurality of training individuals and the training feature vectors, wherein the trained machine learning model inputs a feature vector for a test individual comprising genetic data at the identified subset of SNP loci and generates a prediction of whether the test individual has the phenotypic trait.

20. The method of claim 19, wherein the subset of SNPs with the predictive ability of the phenotypic trait is identified with a genome-wide association study (GWAS) with the plurality of training individuals over the plurality of SNPs, the GWAS comprising:
calculating a p-value score for each SNP based on a positive count of training individuals reported to have the phenotypic trait and a negative count of training individuals reported to not have the phenotypic trait; and
identifying the subset of SNPs based on the p-value scores for the plurality of SNPs being below a threshold p-value score;
